(19) United States
(12) Patent Application Publication
(10) Pub. No.: US 2022/0330601 A1
(43) Pub. Date: Oct. 20, 2022

(54) APPARATUS FOR AND METHOD OF STEAM TREATING OF PLANT FIBRES

(71) Applicant: HAYGAIN LTD, Lambourn, Berkshire (GB)

(72) Inventors: Becky WINDELL, Lambourn, Berkshire (GB); James MILES, Lambourn, Berkshire (GB); Edzo WISMAN, Lambourn, Berkshire (GB)

(73) Assignee: HAYGAIN LTD, Lambourn, Berkshire (GB)

(21) Appl. No.: 17/640,468

(22) PCT Filed: Sep. 4, 2020
(86) PCT No.: PCT/GB2020/052130

§ 371 Date: Mar. 4, 2022

(30) Foreign Application Priority Data
Sep. 6, 2019 (GB) 1912869.3

(51) Int. Cl. A23N 17/00; A61L 2/07; A23K 10/32; A23K 30/10; A23B 7/005; G01K 3/00

(52) U.S. Cl. CPC A23N 17/004; A61L 2/07; A23K 10/32; A23K 30/10; A23N 17/008; A23B 7/0053; G01K 3/005; A61L 2202/14; A23V 2002/00

(57) ABSTRACT

An apparatus for steam treating plant fibres is provided, the apparatus comprising: a steam manifold that is adapted to receive steam from a steam source and to distribute steam to a plurality of lances, the lances being configured to deliver steam into the interior volume of a batch of plant fibres; a temperature sensor for sensing a temperature of the batch of plant fibres, and a controller. The controller is configured to receive a signal from the temperature sensor, and to control the supply of steam to the lances to raise the temperature of the plant fibres to a target temperature. A method of steaming plant fibres is also provided.

### FIELD

The invention relates to an apparatus for and method of steam treating plant fibres, for example animal fodder (such as hay, grasses, herbaceous legumes, tree legumes, silage and crop residues) or industrial fibres (such as industrial hemp fibre). The apparatus is particularly suitable for steam treating batches of plant fibres, which are typically in baled form, which is intended to include bales as well as retaining nets, bags, baskets or similar receptacles.

### BACKGROUND

The primary purpose of steam treating is to kill mesophilic and thermophilic mould spores and bacteria that are either attached to plant fibres or detach when disturbed and become airborne. These airborne particles are commonly assumed as dust spores, together with any living organisms and can include insects and the like. The purpose therefore of treating the plant fibres is to kill all the aforesaid, for example before the plant fibres are processed, or prior to fodder being fed to livestock; thus reducing the risks of creating or aggravating respiratory problems, infections and allergies from such or similar organisms. The problem associated with respiratory conditions, infections and allergies applies to both livestock being fed as well as humans handling the plant fibres or when preparing and feeding the plant fibres to livestock as fodder.

Most forms of livestock are fed predominantly on conserved fodder from manmade bales; in their whole, in part, or detached from the whole bale and inserted in a receptacle such as a net or basket.

Fodder is one of the cheapest and most widely available natural forms of feeding livestock and provides most of the nutrients required. When fodder, such as grasses and crop residues, is cut, it is usually compressed into bales for ease of storage and manoeuvrability. All fodder contains leaf shatter, soil, mesophilic moulds, plant particles, fragments of sundry inorganic materials, bacteria, fungi and fungal spores, insects, and other organisms in varying amounts. When the fodder has been cut and stored additional organisms (thermophilic actinomycetes) are also present. All of this matter is generally classified as dust.

Much of this dust is present in particles of less than 5 microns in diameter (respirable particles) and these particles can cause an allergic reaction within some livestock (e.g. horses and certain goat species). The allergic reaction is precipitated by a hypersensitivity to the respirable particles, which leads to airway inflammation, bronchoconstriction and accumulation of mucoid secretion in the animal's airways.

Clinical signs such as coughing and reduced capacity for exercise are persistent. These conditions include the well-known Recurrent Airway Obstruction (RAO)—also known as Chronic Obstructive Pulmonary Disorder (COPD)—and are responsible for a significant loss of revenue in terms of days in training and reduced performance. Moreover, these respirable particles are the cause of the debilitating condition in humans known as Farmer's Lung, as well as more common hay fevers. Some livestock owners soak their fodder, such as hay, to reduce the number of airborne particles released during feeding.

However, the initial handling of the material usually results in the dust becoming airborne and present in the atmosphere exposing animals and humans alike to hazardous respirable particles. While soaking fodder has proved effective in reducing respirable particle numbers, it does not kill the fungi and bacteria present and thus ingestion of these pathogens still occurs and can lead to other associated problems, particularly in breeding livestock. Furthermore soaking has been scientifically proven to leach some of the nutritional content from the fodder; and produces a post-soak liquid that has a high biological oxygen demand classifying it as an environmental pollutant.

Steam treating may also be beneficial in the processing of plant fibres for use in industrial processes, for example where bales of plant fibres which have been baled and stored after harvesting may benefit from decontamination by steam treating to kill mould spores and bacteria present on the fibres.

### PRIOR ART

Examples of steam treating fodder are described in UK Patent Application GB 2 338 167 A (Meech & Davis).

Another type of fodder steam treatment system is described in UK Patent Application GB 2 387 311 A (Bottomley).

Although the aforementioned systems operated with a reasonable degree of success they suffered from a number of drawbacks.

Another type of hay steamer was made and sold by Happy Horse Products limited and includes a conventional steam generator which delivers steam, via a lance, into loosely packed fodder which is contained in a bag. In the event that the bag is waterproof steam condenses in the bag with the result that there is a build up of hot water condensate in the bag and the aforementioned risk of leaching of nutrients from the fodder.

An example of an apparatus for steam treating fodder in baled form is disclosed in European patent EP2364100B1 of Haygain Limited.

The present invention overcomes problems associated with the aforementioned prior art systems.

### SUMMARY OF THE INVENTION

According to a first aspect of the invention there is provided apparatus for steam treating plant fibres, the apparatus comprising:

a steam manifold that is adapted to receive steam from a steam source and to distribute steam to a plurality of lances, the lances being configured to deliver steam into the interior volume of a batch of plant fibres;

a temperature sensor for sensing a temperature of the batch of plant fibres; and

a controller configured to receive a signal from the temperature sensor, and to control the supply of steam to the lances to raise the temperature of the plant fibres to a target temperature.

The plurality of lances being configured to deliver steam into the interior volume of a batch of plant fibres may advantageously ensure that there is an even distribution of steam throughout the volume of the batch plant fibres. This may ensure that the humidity and temperature of the plant fibres as a whole is raised to a desired level.

The use of a temperature sensor and a controller configured to receive a signal from the temperature sensor advantageously allows improved control of the steaming process compared to the devices known in the prior art. Whereas in prior art devices the steaming process has been relatively simple and not actively controlled, the controller and temperature sensor of the present invention allow improved control of the temperature to which the plant fibres are heated during steaming. This may be particularly advantageous when steaming temperature-sensitive plant fibres, where excessive temperatures may cause undesirable degradation of the fibres.

The temperature sensor may preferably be provided on a probe insertable into the batch of plant fibres during steaming.

The apparatus may comprise a valve or valve means controllable to prevent or allow the passage of steam from the steam source to the lances, and in which the valve or valve means are controllable by the controller.

The use of a valve or valve means between the steam source and the lances may advantageously allow the controller to stop and start the flow of steam to the lances during steaming.

The valve or valve means may allow the controller to generate steam pressure in the steam source prior to opening the valve or valve means, so that steam is delivered to the lances and into the plant material at a pressure greater than atmospheric pressure. This may advantageously mean that the steam is delivered at high pressure and the plant material is heated to a target temperature more quickly than was possible with prior art devices. By heating the plant material more quickly, this may advantageously reduce the length of time during which the steam must be delivered to the plant fibres in order to reach a temperature sufficient to kill spores. This may in turn allow the plant fibres to be sterilised more quickly, and to become less damp during the steaming process thanks to the reduced time in contact with steam. This may be a particular benefit when steaming plant fibres, which degrade in the presence of excessive moisture.

The valve or valve means may also allow improved control of the steaming process by the controller, as the controller can stop and start the steam flow as desired, for example to maintain the plant fibres at the target temperature or within a target temperature range.

The controller may comprise a temperature control feedback loop. The controller may be configured to control the steam supply so that the temperature of the batch of plant fibres is maintained at a target temperature for a predetermined period of time.

The use of a temperature feedback loop may advantageously allow the controller to receive temperature signals from the temperature sensor, and to adjust the steam supply dependent on the sensed temperature of the plant fibres. This may allow improved temperature control of the plant fibres during steaming, which may be particularly beneficial where the plant fibres are prone to degrade when subjected to high temperatures.

The controller may be configured to stop the supply of steam when the sensed temperature exceeds a predetermined level, or when the temperature of the plant fibres has been maintained at the target temperature for a predetermined period of time.

The controller may thus operate in a temperature control mode where the temperature of the plant fibres are limited to a predetermined maximum temperature.

The controller may be programmable by a user.

The controller may be controllable to set a target temperature, and/or a target humidity, and/or a target steaming time, and/or a target steaming pressure.

The controller may be built into the apparatus or communicate with the apparatus, for example, via a wired or wireless interface. In certain embodiments, the controller may be a mobile phone, a tablet computer, or any other handheld control device. For example, an application may be downloaded onto a mobile device allowing the device to be used as a controller. The controller can be configured to provide a user interface that allows users to input desired configurations (e.g., a target temperature, target humidity, target steaming time, and/or a target steaming pressure).

In more general terms, the controller may comprise a data processing system and/or a computer program product, for example as described in more detail further below.

The apparatus may comprise a container configured to contain the plurality of lances and, in use, the batch of plant fibres.

The container is preferably thermally insulated. This advantageously improves the thermal efficiency of the apparatus by reducing heat loss from the container.

By providing at least the lances in a container which can receive a batch of plant fibres for steaming, the container may confine the steam during steaming, enabling the plant fibres to reach a higher temperature and/or humidity more quickly than would be possible outside a container.

The container may be sealable in a gas-tight configuration, so that steam pressure inside the container can be increased above atmospheric pressure.

This may advantageously allow the temperature of the plant fibres to be increased rapidly to a level sufficient to kill mould spores. Plant fibres may therefore be sterilized with less time in contact with moist steam, which may lead to less condensation and less moisture absorbed into the plant fibres during steaming. This may be beneficial when steam cleaning certain types of plant fibres where it is undesirable to raise the moisture level too high.

In a preferred embodiment, the container comprises an openable vent, the openable vent being controllable by the controller between an open state in which steam is vented from the interior of the container to the atmosphere, and a closed state in which steam cannot pass through the vent.

The controller may be programmable to open the vent as soon as steaming is complete. For example, the controller may open the vent as soon as the plant fibres have reached the target temperature, or to open the vent after the plant fibres have been held at the target temperature for a predetermined period of time. Opening the vent may advantageously allow steam to escape from the container, reducing the amount of condensation that accumulates on the container walls and in the plant fibres.

The vent may be continuously adjustable between its closed and open states to allow the controller to control the pressure of the steam in the container.

The apparatus may comprise a pressure sensor configured to sense a pressure inside the container, and the controller may be configured to receive signals from the pressure sensor.

The controller may be controllable to set a target steaming pressure. The controller preferably comprises a pressure control feedback loop, and is configured to control the steam supply so that the pressure of steam in the container is increased to the target steaming pressure.

The controller may be programmed to stop the supply of steam, or to open the vent, when the pressure in the container exceeds a predetermined level or reaches the target pressure.

The apparatus may comprise a humidity sensor configured to sense a humidity inside the container, and the controller may be configured to receive signals from the humidity sensor.

The humidity sensor may be provided on a probe insertable into the batch of plant fibres during steaming, so that the humidity sensor might measure moisture content inside the batch during steaming, or the humidity sensor may be provided on a wall of the container containing the batch of plant fibres.

The controller may be controllable to set a target humidity. The controller may comprise a humidity control feedback loop, and be configured to control the steam supply so that the humidity in the container is increased to the target humidity.

The controller may be programmed to stop the supply of steam when the sensed humidity exceeds a predetermined level or reaches the target humidity.

The controller may be configured to determine that steaming is complete based on signals from the humidity sensor. For example, the controller may open the vent as soon as the plant fibres have reached the target humidity, or open the vent after the plant fibres have been held at the target humidity for a predetermined period of time.

By sensing the temperature and the pressure and/or humidity in the container during steaming, the controller may advantageously control the steam supply and optionally the vent to control the steaming conditions of the plant fibres. Steaming temperatures, pressures and humidities may thus be controlled to a greater extent than has been possible with prior art devices. This may allow the apparatus to provide different steaming conditions for different batches of plant fibres.

In combination with the valve or valve means and/or the vent, the controller may ensure that the target temperature, and/or a target humidity, and/or a target steaming time, and/or a target steaming pressure is maintained for a predetermined period of time.

For example, when steaming fibres such as hay or industrial hemp fibre, it may be desirable to impart a high moisture content into the fibres. In this case, the controller may be programmed to steam the fibres for a long period of time, preferably at a low steam pressure. This may allow time for moisture to condense onto the plant fibres during steaming, greatly increasing the moisture content of the plant fibres.

Alternatively, when steaming fibres that may degrade in damp conditions, the controller may be programmed to deliver high pressure steam to the container with the vent closed. This may increase the temperature of the plant fibres rapidly to a temperature sufficient to kill microorganisms. After a predetermined steaming period, the vent may then be opened to vent the steam from the container, to reduce the quantity of condensation that is left in the plant fibres.

The apparatus may comprise a steam generator configured to deliver steam to the manifold, such that the steam generator forms the steam source. For example, a steam generator may be provided inside the container, or integrated with the container body or housing. In preferred embodiments, the steam generator may be housed in the base of the container, or in a wall of the container.

In a preferred embodiment, the steam generator and the manifold are provided in a shared housing. The outlet of the steam generator may form the inlet of the manifold.

By providing the steam generator and the manifold in the same housing, the distance that steam must travel between the steam generator and the manifold may be reduced or minimised. This may advantageously allow steam to be delivered to the lances at a higher pressure and/or temperature than is possible with a “remote” steam generator, as the steam does not have time to cool down, lose pressure and condense as it travels through piping from the steam generator.

The steam generator is preferably configured to deliver steam to the lances at a pressure greater than atmospheric pressure, for example at least 1.2 bar, or at least 1.4 bar, or at least 1.6 bar, or at least 2 bar.

By delivering steam at a raised pressure, the plant fibres may be heatable more quickly, with less time for moisture to condense in the plant fibres during steaming.

The controller may be programmed to operate in a defrost mode for defrosting and re-hydrating frozen plant fibres, in which the controller delivers steam to raise the temperature of a frozen batch of plant fibres to a target temperature range of between 3° C. and 8° C., preferably between 4° C. and 6° C., or 4° C. and 5° C.

In defrost mode, the controller may preferably deliver steam to the batch of plant fibres until the moisture content of the batch of plant fibres is between 8% and 14%, preferably between 10% and 12% moisture content.

The controller may be programmed to operate in a steam cleaning mode for killing bacteria on plant fibres, in which controller delivers steam to raise the temperature of a batch of plant fibres to a target temperature range of between 90° C. and 104° C.

In steam cleaning mode, the controller may preferably be configured to deliver steam to raise the temperature of the batch of plant fibres to a temperature of at least 90° C. for a period of at least 10 minutes, or at least 15 minutes, or at least 20 minutes. Lower temperatures and steaming durations may be insufficient to ensure harmful microorganisms have been killed by steaming.

The controller may be configured to deliver steam to raise the temperature of the batch of plant fibres to a maximum temperature of 104° C. for a maximum of 30 minutes. Higher temperatures and time durations may cause deterioration of the plant fibres during steaming.

Preferably, the controller may deliver steam to raise the temperature of the batch of plant fibres to between 90° C. and 104° C. for between 10 and 30 minutes, or between 12 and 25 minutes, or between 15 and 20 minutes. Particularly preferably, the controller may deliver steam to raise the temperature of the batch of plant fibres to between 100° C. and 104° C. for between 10 and 30 minutes.

When the controller is programmable by a user, a user may be able to select the mode of operation of the controller such that the controller operates in the defrost mode or the steam cleaning mode.

In the steam cleaning mode, the controller is preferably programmable so that the user can select a target temperature, target pressure and/or target humidity of the batch of plant fibres for the controller. The user may also program the controller to set the duration of the steaming time. The user may make these selections based on the type and starting quality of the plant fibres.

The controller may be pre-programmed to be operable in several pre-set modes, such as a defrost mode, and one or more steam cleaning modes. For example, for steam cleaning plant fibres, the user may select a first steam cleaning mode with a target temperature of around 90° C. For plant fibres that are particularly humidity sensitive, the user may select a second steam cleaning mode with a higher target temperature of more than 104° C. This may allow the plant fibres to be sterilised more quickly and to become less damp during the steaming process as the amount of time that the plant fibres are in contact with steam may be reduced.

The controller may be programmable to operate the apparatus in a very high temperature steam cleaning mode with a target temperature of greater than 104° C. In this mode, the controller may control the apparatus to raise the temperature of the plant fibres to a target temperature of between 104° C. and 125° C., or between 108° C. and 120° C., or between 110° C. and 115° C. This mode may be particularly suitable for sterilising animal fodder by killing certain microorganisms that are not killed at lower temperatures. In this mode, the apparatus may preferably deliver steam under increased pressure to quickly raise the temperature of the plant fibres to the desired high target temperature without causing the plant fibres to become too wet.

The “plant fibres” steamable with the apparatus may be animal fodder, such as hay or haylage, straw or alfalfa. Alternatively, the plant fibres may be industrial fibres such as industrial hemp.

In a preferred embodiment, the plant fibres may be selected from a group consisting of animal fodder or industrial hemp. Thus, the apparatus may be an apparatus for steam treating animal fodder, or an apparatus for steam treating industrial hemp.

A batch of plant fibres may, for example, include plant fibres in baled form, for example plant fibres compressed into bales, or plant fibres held in nets, mesh bags or other containers or receptacles.

The lances may all be the same length, or height.

Alternatively, the plurality of lances may have different lengths. Lances of different lengths arranged on the same manifold may advantageously provide improved steam distribution throughout a batch of plant fibres by distributing steam to different areas within the batch. This may be particularly beneficial where the plant fibres are provided in a large bale, so that steam must percolate throughout a large volume of plant fibres. This ensures even steaming throughout the volume of plant fibres. For example, a large rectangular or round bale of animal fodder may have a length greater than 1449 millimetres, a depth 815 millimetres and/or a height greater than 750 millimetres. In order to distribute steam throughout the volume of a bale this size, the apparatus preferably comprises lances with different lengths which extend to different heights within the bale. In an apparatus configured to steam a 750 mm-thick bale of fodder, the lengths of the lances may for example vary between 50 mm and 700 mm in height, or between 100 mm and 650 mm in height, so that different lances extend different distances into the bale.

The apparatus may comprise a lance with a first length, a lance having a second length greater than the first length, and a lance having a third length greater than the second length.

In a preferred embodiment, the apparatus may comprise at least two lances with a first length, and at least one lance having a second length greater than the first length. The apparatus may further comprise at least one lance having a third length greater than the second length. The third length may be at least twice the first length. These lances may allow steam to be distributed at different heights within a batch of plant fibres, for example a bale of plant fibres, so that steam is distributed throughout the batch.

Ideally, the steam manifold, in use, is arranged to impale a bale of plant fibre, by placing the bale thereon, thus the weight of the bale assists in the process of ensuring the lances penetrate into the bulk of the bale.

The manifold may be adapted to rest on the ground or floor of an area and a bale may be placed on it or the manifold may be driven into a bale. Whichever technique is used there is a single action involved in steam treating the plant fibres and once treated, any baler twine or other binding is cut and the fibres can be distributed. Therefore, the process is quick to implement and a user is free to do other things whilst a batch of plant fibres is steam treated.

The apparatus is preferably formed from a strong and heat resistant material, such as stainless steel, other metals or synthetic plastics material, which is able to withstand temperatures in excess of 110 degrees Centigrade.

The manifold ideally includes a plurality of lances, which may be in the form of prongs or spikes, each being adapted to impale a batch of plant fibres so that they penetrate deep into the batch, thereby enabling steam to pass into the bulk of the batch and percolate from the centre outwards. This ensures deep and thorough heating—and thus steam treatment—of the fibres.

There may be two, but ideally there are three, four, five or more lances arranged in an array so that the lances are in a form that enhances even distribution of steam throughout the batch. Again ensuring deep and thorough heating—and thus steam treatment—of the plant fibres. In an example, the array may comprise at least two lances of the first length, at least one lance of the second length and at least one lance of the third length, the lengths as described above.

The size of the manifold, and the number of lances in the array, may be chosen to correspond to the capacity of the container, and therefore the volume of plant fibres the apparatus is configured to steam. Alternatively, the apparatus may comprise a plurality of manifolds. Each manifold may comprise a plurality of lances arranged in an array, as described above.

Ideally, the apparatus includes a heater which has an immersion element and is adapted for use with either 240 Volts or 110 Volts. The heater generates steam in the well-known manner.

In a second aspect, the invention may provide a method of steaming plant fibres, comprising the steps of:

inserting a plurality of lances into a batch of plant fibres;

delivering steam from a steam source, through a manifold, and out of the lances into the batch of plant fibres;

sensing a temperature of the batch of plant fibres; and controlling the supply of steam to the lances in response to the sensed temperature, to raise the temperature of the plant fibres to a target temperature.

The method of steaming plant fibres may comprise the use of the apparatus described above as the first aspect of the invention. Thus, the step of controlling the supply of steam may be carried out by a controller.

In a preferred embodiment of the method, steam may be delivered to the plant fibres at a pressure greater than atmospheric pressure, for example at least 1.2 bar, or at least 1.4 bar, or at least 1.6 bar, or at least 2 bar.

The steam supply may be controlled so that the temperature of the batch of plant fibres is maintained at a target temperature for a predetermined period of time.

The supply of steam may be stopped when the sensed temperature exceeds a predetermined level.

In a preferred embodiment, the plurality of lances and the batch of plant fibres are placed in a container before steam is delivered from the steam source. The container may be sealed in a gas-tight configuration while the steam is delivered to the plant fibres, so that steam pressure inside the container increases above atmospheric pressure.

The method may comprise the step of sensing a pressure inside the container, and/or sensing a humidity inside the container.

The method may comprise the step of controlling the supply of steam to reach a predetermined steam pressure inside the container.

The method may comprise the step of opening, or partially opening, a vent in the container to vent steam from the interior of the container to the outside atmosphere when the pressure, or the temperature, or the humidity in the container exceeds a predetermined level.

In a further aspect, the invention may provide a method of defrosting and re-hydrating frozen plant fibres, comprising the method of the second aspect of the invention,

in which the supply of steam is controlled to raise the temperature of a frozen batch of plant fibres to a target temperature range of between 3° C. and 8° C., preferably between 4° C. and 6° C., or 4° C. and 5° C.

In order to re-hydrate frozen plant fibres, steam is preferably supplied to the batch of plant fibres until the moisture content of the batch of plant fibres is between 8% and 14%, preferably between 10% and 12% moisture content.

In a further aspect, the invention may provide a method of steam cleaning plant fibres, comprising the method of the second aspect of the invention, in which the supply of steam is controlled to raise the temperature of a batch of plant fibres to a target temperature range of between 90° C. and 104° C.

In order to steam clean the plant fibres the supply of steam may be controlled to raise the temperature of the batch of plant fibres to a temperature of at least 90° C. for a period of at least 10 minutes, or at least 15 minutes, or at least 20 minutes.

The supply of steam may be controlled to raise the temperature of the batch of plant fibres to a maximum temperature of 104° C. for a maximum of 30 minutes.

In a further aspect, the invention may provide an apparatus for steam treating plant fibres, the apparatus comprising:

a steam generator;

a steam manifold that is adapted to receive steam from the steam generator and to distribute steam to a plurality of lances, the lances being configured to deliver steam into the interior volume of a batch of plant fibres;

a housing defining a container for the plurality of lances and, in use, the batch of plant fibres;

in which the steam generator is provided in the same housing as the container.

The housing defines a container suitable for receiving the batch of plant fibres, and the lances are positioned inside the container so that in use they deliver steam into the interior volume of a batch of plant fibres received in the container. For example, the housing may be a box or chest in which the interior volume of the box defines the container for the plant fibres.

Preferably, the housing is a thermally insulated chest. Particularly preferably, the housing may be configured to house the steam generator in a lower portion of the housing, such that the container for receiving the batch of plant fibres is defined in an upper portion of the housing, above the steam generator.

The steam generator may be provided inside the container, or integrated with the container housing. In preferred embodiments, the steam generator may be integrated into a base of the container housing, or in a wall of the container housing.

In a preferred embodiment, the steam manifold is also provided in the housing. The outlet of the steam generator may form the inlet of the manifold.

By providing the steam generator in the same housing as the container, the lances and the steam manifold, the distance that steam must travel between the steam generator and the manifold may be reduced or minimised. This may advantageously allow steam to be delivered to the lances at a higher pressure and/or temperature than is possible with a “remote” steam generator, as the steam does not have time to cool down, lose pressure and condense as it travels through piping from the steam generator.

The steam generator is preferably configured to deliver steam to the lances at a pressure greater than atmospheric pressure, for example at least 1.2 bar, or at least 1.4 bar, or at least 1.6 bar, or at least 2 bar.

By delivering steam at a raised pressure, the plant fibres may be heatable more quickly, with less time for moisture to condense in the plant fibres during steaming.

The apparatus may comprise a temperature sensor for sensing a temperature of the batch of plant fibres, and/or a humidity sensor for sensing a humidity inside the container. The apparatus may further comprise a controller configured to receive a signal from the temperature sensor and/or humidity sensor, and to control the supply of steam to the lances to heat the plant fibres to a target temperature and/or humidity.

Further features of this aspect are described in relation to the first aspect of invention.

The features described above in relation to one aspect of the invention are applicable to any of the other aspects of the invention.

Preferred embodiments of the invention will now be described, by way of examples only, and with reference to the Figures in which:

### BRIEF DESCRIPTION OF THE FIGURES

FIG. 1 is an overall diagrammatical view of one embodiment of the invention and illustrates the principle of operation;

FIG. 2A is an overall view of an example of a manifold and lances;

FIG. 2B is an overall view of an alternative embodiment of manifold and lances having different lengths;

FIG. 3 is a partially transparent illustration of a first embodiment of the invention in use;

FIG. 4 is a partially transparent illustration of a second embodiment of the invention in use; and

FIG. 5 is a schematic diagram of a further embodiment of the invention.

### DETAILED DESCRIPTION OF PREFERRED EMBODIMENTS OF THE INVENTION

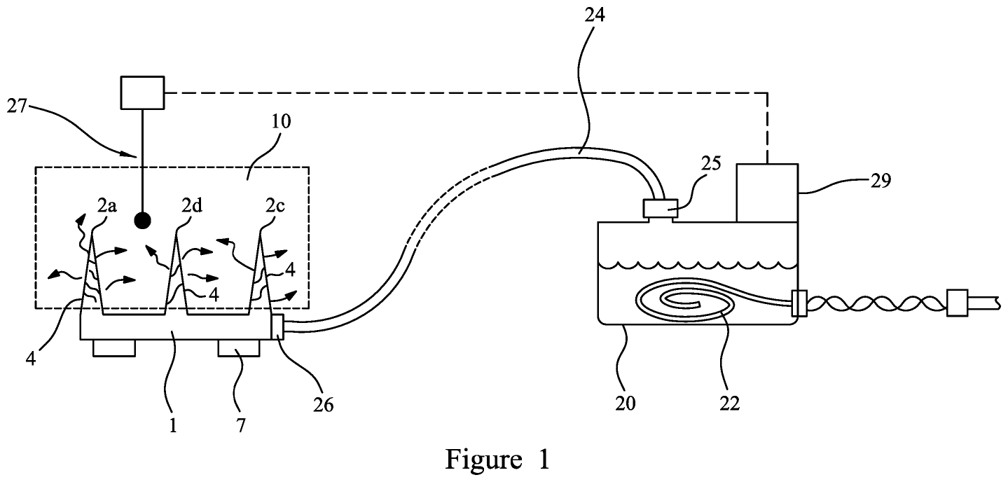

Referring to FIG. 1 there is shown in general an apparatus for steam treatment of plant fibres comprising a water reservoir 20 in which is located a heating element 22. The water reservoir, or steam generator, can also be a sealed vessel and thus capable of heating water to more than its normal boiling point. A high pressure flexible hose 24, which is ideally insulated, conducts steam from the reservoir to a manifold, ideally via a flexible or universal joints 25 and 26. The manifold 1 distributes the steam into a batch of plant fibres 10.

A temperature sensor probe 27 is provided to measure a temperature inside the batch of plant fibres during steaming, and to communicate temperature signals to a controller 29.

The reservoir or steam generator optionally includes conventional safety equipment such as thermostatic settings, boil dry warning and residual current detectors (RCD) for use in damp and outdoor environments.

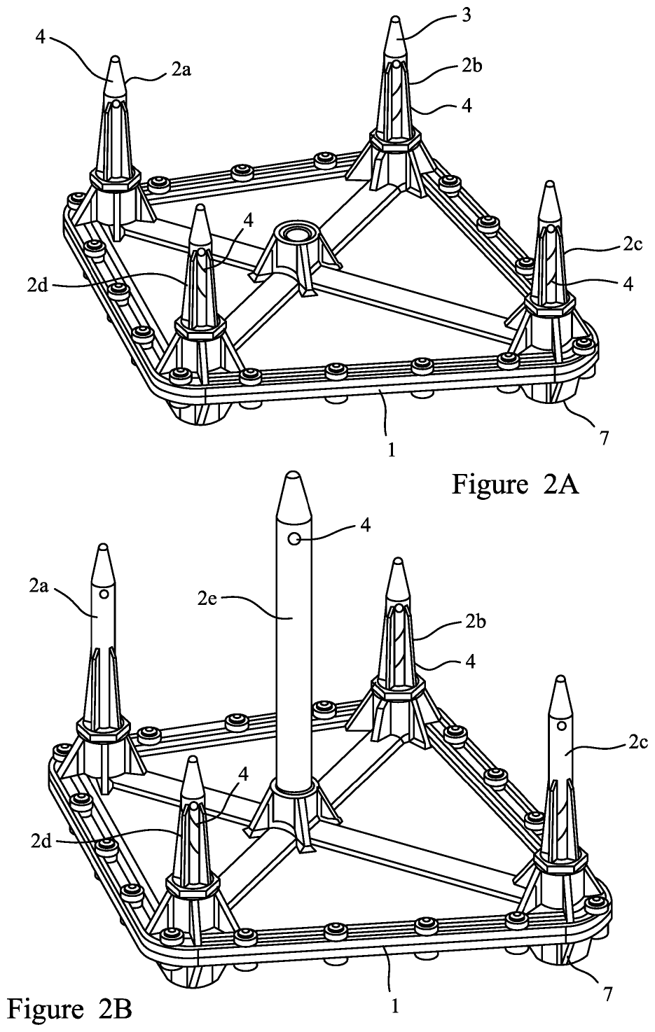

FIG. 2 shows a manifold, which is in the form of a generally square frame and has passages formed therein for distribution of steam. The manifold 1 is fitted with a number of substantially vertical lances 2, which are in communication with the passages. Each lance 2 has a pointed end 3 for ease of penetration into a batch of plant fibre 10. Lances 2 have apertures 4, extending a proportion of their length, for the release and distribution of steam and condensed steam into the centre of the plant fibres.

The supply of steam may be switched on or off by the controller 29. The controller is preferably programmable by a user, and is configured to control steam supply to the plant fibres, and therefore to control the steaming temperature and duration of the steaming.

The controller may be built into the apparatus or communicate with the apparatus, for example, via a wired or wireless interface. In certain embodiments, the controller may be a mobile phone, a tablet computer, or any other handheld control device. For example, an application may be downloaded onto a mobile device allowing the device to be used as a controller. The controller can be configured to provide a user interface that allows users to input desired configurations (e.g., a target temperature, target humidity, target steaming time, and/or a target steaming pressure).

The controller may comprise a data processing system, which may include one or more processors (e.g., a general purpose microprocessor and/or one or more other data processing circuits, such as an application specific integrated circuit (ASIC), field-programmable gate arrays (FPGA's), and the like); a network interface for connecting the controller to a network; and a local storage unit, which may include one or more non-volatile storage devices and/or one or more volatile storage devices (e.g., random access memory (RAM)). In embodiments where the controller includes a general purpose microprocessor, a computer program product may be provided. The computer program product may include a computer readable medium (CRM) storing a computer program comprising computer readable instructions. The CRM may be a non-transitory computer readable medium, such as, but not limited to magnetic media (e.g., a hard disk), optical media (e.g., a DVD), memory devices (e.g., random access memory), and the like. In some embodiments, the computer readable instructions are configured such that when executed, they cause the controller to perform tasks described herein. In other embodiments, the controller may be configured to perform tasks described herein without the need for code. For example, the data processing system may consist merely of one or more ASICs.

The apertures 4 may be vertically disposed or they may be in the form of slits or slots, extending lengthwise or helically about circular lances 2. Alternatively, the apertures may take the form of holes formed in the lances so that the steam and condensed steam permeate outwards to the extremities of the batch of plant fibres ensuring full effectiveness and contact of the steam throughout the fibres. It has been found that with use of the manifold the temperature of a bale may exceed 100 degrees Centigrade.

As a result of the lances 2a, 2b, 2c and 2d, steam is introduced into the centre of the bale or receptacle of the fibres by placing the batch of plant fibres 10 onto the manifold 1. Alternatively, the manifold 1 can be forced into a bale of plant fibres 10 from the side or above. In whichever orientation the lances penetrate so as ensure steam reaches all of the bale. If the manifold is arranged to rest on the ground during operation, an optional foot or feet 7 may be provided or formed on the manifold to prevent damage to it, for example by shock loading that may occur when a bale is dropped onto the lances.

In an alternative embodiment one or more steam distribution manifolds 1 of various lengths (to accommodate different size bales and receptacles to ensure effective central penetration) is provided. Lances 2 may be of different lengths and optionally these may be removable and fitted onto a common manifold. Thus for example in the event of damage to a lance, it may be removed and replaced with a similar lance. Alternatively, where the manifold is required for use with larger bales, longer lances may be fitted to the manifold.

FIG. 2B shows an embodiment in which five lances 2a, 2b, 2c, 2d and 2e are connected to a manifold 1. In order to provide good distribution of steam at different heights in a bale of plant fibres, the lances have different lengths, so that they extend to different heights above the manifold 1. As shown in FIG. 2B, two lances 2b and 2d on opposite sides of the manifold have a first length or height, two lances 2a and 2c on opposite sides of the manifold have a second length or height, which is greater than the first length, and a lance 2e in the centre of the manifold has a third length or height which is greater than the lengths of the other lances. Apart from the lance lengths, the design of each lance is substantially the same as described above in relation to FIG. 2A. When a large bale of plant fibres is placed on the manifold 1 for steaming, the lances will penetrate the bale so that the tips of the lances, and therefore the steam apertures, extend to different heights inside the bale. This means that steam is delivered to different points inside the bale, providing more uniform steaming throughout the large bale than would be possible with lances of uniform length.

Steam and condensed steam permeate through the plant fibres increasing the temperature of the fibres to between 90 and 105 degrees Centigrade (depending upon ambient temperature) killing thermophilic and mesophilic mould spores and other living organisms as mentioned above and effectively steam treating the fibres as well as dampening dust spores thus restricting their ability to become airborne.

The steam is distributed from the reservoir, via the hose and through the lances and where the steam condenses the water content is absorbed, in the majority, by the plant fibres leaving it damp. As the moisture content within the plant fibres increases, the temperature rises exponentially due to the increased efficiency of water as a heat conducting medium within the fibres, compared to air in the fibres' dry state.

In prior art devices, the lack of control system meant that fibres were typically steamed continuously for a long period, during which the fibres absorbed moisture and the temperature of the fibres increased exponentially. The temperature probe 27 and controller of the present device, however, allow more precise control so that the plant fibres can be heated to and maintained at a desired target temperature for the desired duration.

Depending on the programming of the controller, the plant fibres may be exposed to continuous steam from the apparatus, or the steam supply may be intermittently stopped and started to maintain the fibres at a desired temperature, for example.

The controller may be programmed to operate in a steam cleaning mode for killing bacteria on plant fibres. In order to kill microorganisms the fibres, for example animal fodder, may typically be heated to a temperature of at least 90 degrees centigrade for not less than 10 minutes.

Alternatively, the controller may be programmed to operate in a defrost mode for defrosting and re-hydrating frozen plant fibres, in which the controller delivers steam to raise the temperature of a frozen batch of plant fibres to a target temperature range of between 3° C. and 8° C.

In defrost mode, the controller may delivers steam to the batch of plant fibres until the moisture content of the batch of plant fibres is between 8% and 14%, preferably between 10% and 12% moisture content.

This defrost mode may be particularly suitable, for example, for defrosting industrial hemp which has been frozen for storage and transport.

A user can select the mode of operation of the controller such that it is defrost mode or a steam cleaning mode.

The aforementioned apparatus can be used either in open space or within an enclosed environment, such as horse box, stable or barn.

In a preferred embodiment the manifold 1 and lances 2 are provided inside a designated sealable container 30, which is preferably insulated and gas-tight so that steam pressure may be built up within the container.

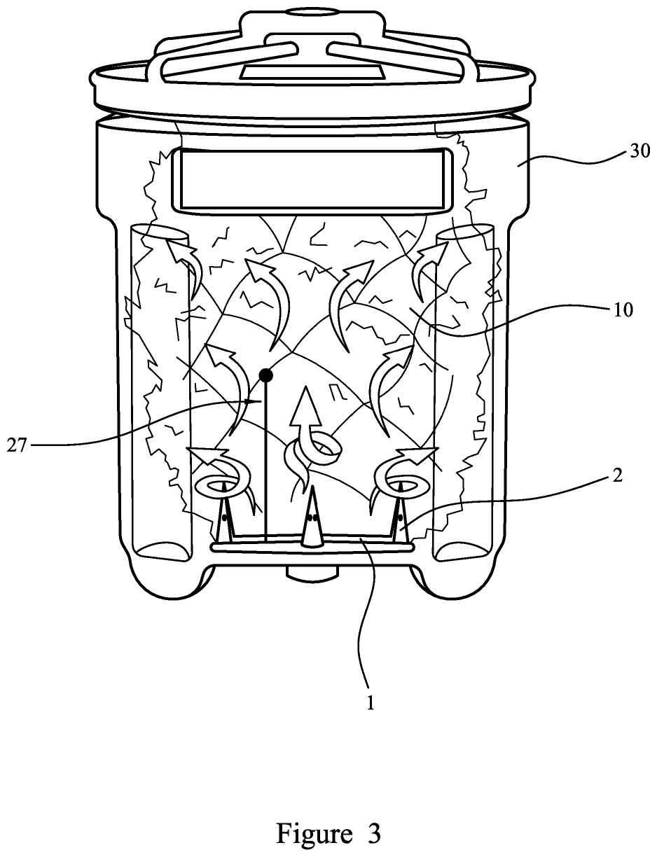

FIG. 3 illustrates a first embodiment of a steaming apparatus in which a batch of plant fibres contained in a net 10 are steamed in a container 30. A manifold 1 is positioned in the base of the container 30, so that in use the batch of plant fibres is lowered down onto the lances 2 so that they penetrate into the batch of fibres. A temperature probe 27 also projects upwards from the base of the container so that it penetrates into the batch of fibres.

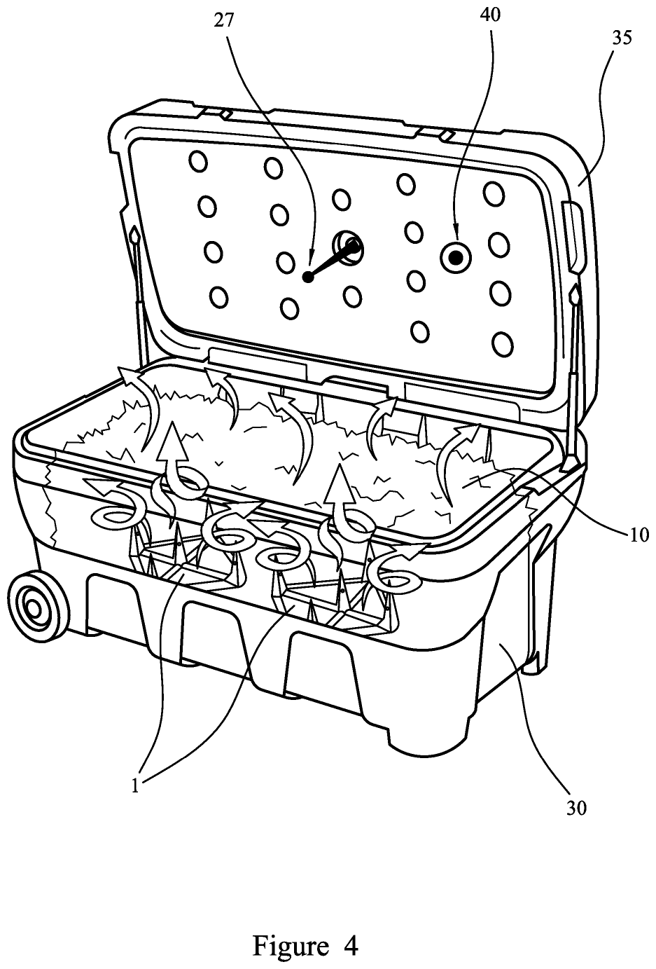

FIG. 4 shows an alternative embodiment of the steaming apparatus including a larger container 30 which contains two manifolds 1 in its base. In this embodiment, the temperature probe 27 is provided on the lid 35 of the container.

When closed, the lid 35 preferably forms a gas-tight seal with the container walls so that the steam pressure in the container can be increased to a desired pressure. A pressure sensor (not shown) is provided in a container wall, which communicates pressure signals to the controller.

A closable vent 40 is provided in the container lid 35 for releasing steam from the interior of the container. Opening and closing of the vent is controllable by the controller, either in response to a threshold pressure being reached, or to release steam from the container at the end of the desired steaming time.

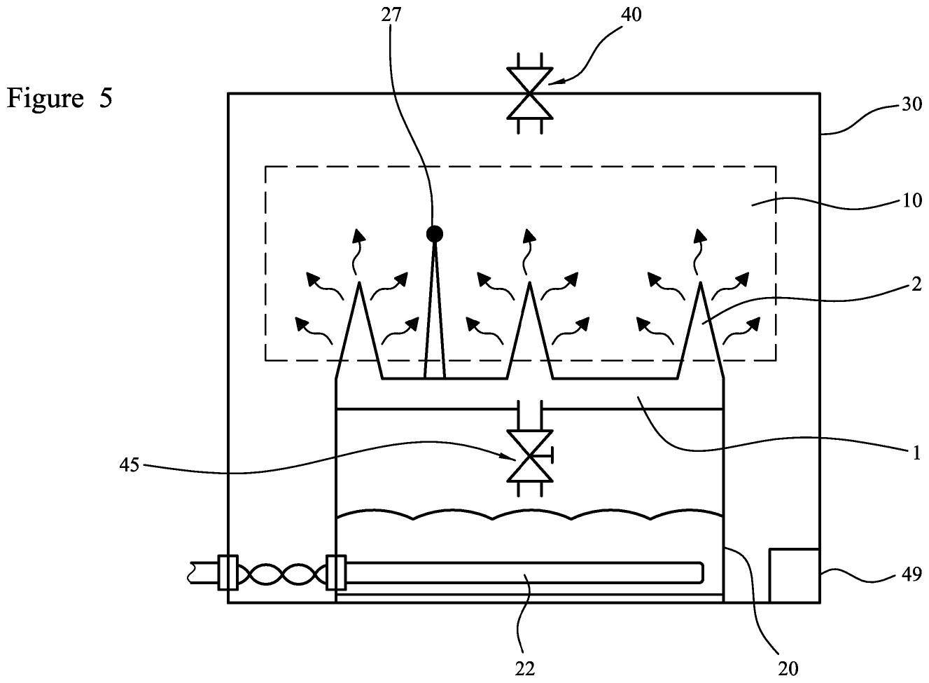

In the embodiment of the apparatus shown in FIG. 5, the manifold 1 is positioned directly above the boiler 20, with a valve 45 controlling the flow of steam from the boiler to the manifold. The opening and closing of the valve 45 is controllable by a controller 49. As the boiler and the manifold are adjacent to one another, high pressure steam may be generated in the boiler and supplied to the manifold without having the chance to cool down and condense in a connecting hose 24. This may be particularly preferred for supplying high pressure steam to the batch of plant fibre 10 in the interior of the container 30, as it may allow rapid heating of the plant fibre to kill spores and microorganisms. Once the plant fibre has been heated to sufficient temperature for long enough, the controller preferably opens the vent 40 to release the steam in the container and reduce the amount of condensation left in the plant fibres.

1. An apparatus for steam treating plant fibres, the apparatus comprising:
a steam manifold that is adapted to receive steam from a steam source and to distribute steam to a plurality of lances, the lances being configured to deliver steam into the interior volume of a batch of plant fibres;
a temperature sensor for sensing a temperature of the batch of plant fibres; and
a controller configured to receive a signal from the temperature sensor, and to control the supply of steam to the lances to raise the temperature of the plant fibres to a target temperature.

2. An apparatus according to claim 1, in which the apparatus comprises valve means controllable to prevent or allow the passage of steam from the steam source to the lances, and in which the valve means are controllable by the controller.

3. An apparatus according to claim 1 or 2, in which the controller comprises a temperature control feedback loop, and is configured to control the steam supply so that the temperature of the batch of plant fibres is maintained at a target temperature for a predetermined period of time.

4. An apparatus according to claim 1, 2 or 3, in which the controller is configured to stop the supply of steam when the sensed temperature exceeds a predetermined level.

5. An apparatus according to any preceding claim, in which the apparatus comprises a container configured to contain the plurality of lances and, in use, the batch of plant fibres.

6. An apparatus according to claim 5, in which the container is sealable in a gas-tight configuration, so that steam pressure inside the container can be increased above atmospheric pressure.

7. An apparatus according to claim 5 or 6, in which the container comprises an openable vent, the openable vent being controllable by the controller between an open state in which steam is vented from the interior of the container to the atmosphere, and a closed state in which steam cannot pass through the vent.

8. An apparatus according to claim 5, 6 or 7, in which the apparatus comprises a pressure sensor configured to sense a pressure inside the container, and in which the controller is configured to receive signals from the pressure sensor.

9. An apparatus according to claim 8, in which the controller comprises a pressure control feedback loop, and is configured to control the steam supply so that the pressure of steam in the container is increased to a target level.

10. An apparatus according to claim 8 or 9, in which the controller is programmed to stop the supply of steam, or to open the vent, when the pressure in the container exceeds a predetermined level.

11. An apparatus according to any preceding claim, in which the apparatus comprises a humidity sensor configured to sense a humidity inside the container, and in which the controller is configured to receive signals from the humidity sensor.

12. An apparatus according to claim 11, in which the controller comprises a humidity control feedback loop, and is configured to control the steam supply so that the humidity in the container is increased to a target level.

13. An apparatus according to claim 11 or 12, in which the controller is programmed to stop the supply of steam when the sensed humidity exceeds a predetermined level.

14. An apparatus according to any preceding claim, in which the apparatus comprises a steam generator configured to deliver steam to the manifold.

15. An apparatus according to claim 14, in which the steam generator and the manifold are provided in a shared housing.

16. An apparatus according to claim 14 or 15, in which the steam generator is configured to deliver steam to the lances at a pressure greater than atmospheric pressure, for example at least 1.2 bar, or at least 1.4 bar, or at least 1.6 bar, or at least 2 bar.

17. An apparatus according to any preceding claim, in which the controller is programmed to operate in a defrost mode for defrosting and re-hydrating frozen plant fibres, in which controller delivers steam to raise the temperature of a frozen batch of plant fibres to a target temperature range of between 3° C. and 8° C., preferably between 4° C. and 6° C., or 4° C. and 5° C.

18. An apparatus according to claim 17, in which the controller delivers steam to the batch of plant fibres until the moisture content of the batch of plant fibres is between 8% and 14%, preferably between 10% and 12% moisture content.

19. An apparatus according to any preceding claim, in which the controller is programmed to operate in a steam cleaning mode for killing bacteria on plant fibres, in which controller delivers steam to raise the temperature of a batch of plant fibres to a target temperature range of between 90° C. and 104° C.

20. An apparatus according to claim 19, in which the controller is configured to deliver steam to raise the temperature of the batch of plant fibres to a temperature of at least 90° C. for a period of at least 10 minutes, or at least 15 minutes, or at least 20 minutes.

21. An apparatus according to claim 19 or 20, in which the controller is configured to deliver steam to raise the temperature of the batch of plant fibres to a maximum temperature of 104° C. for a maximum of 30 minutes.

22. An apparatus according to any preceding claim, in which at least two of the lances have different lengths.

23. A method of steaming plant fibres, comprising the steps of:
inserting a plurality of lances into a batch of plant fibres;
delivering steam from a steam source, through a manifold, and out of the lances into the batch of plant fibres;
sensing a temperature of the batch of plant fibres; and
controlling the supply of steam to the lances in response to the sensed temperature, to raise the temperature of the plant fibres to a target temperature.

24. A method according to claim 23, in which the steam is delivered to the plant fibres at a pressure greater than atmospheric pressure, for example at least 1.2 bar, or at least 1.4 bar, or at least 1.6 bar, or at least 2 bar.

25. A method according to claim 23 or 24, in which the plant fibres are provided as a bale of plant fibres, or as a batch in a net or mesh bag, or container.

26. A method according to claim 23, 24 or 25, in which the steam supply is controlled so that the temperature of the batch of plant fibres is maintained at a target temperature for a predetermined period of time.

27. A method according to any of claims 23 to 26, in which the supply of steam is stopped when the sensed temperature exceeds a predetermined level.

28. A method according to any of claims 23 to 27, in which the plurality of lances and the batch of plant fibres are placed in a container.

29. A method according to claim 28, in which the container is sealed in a gas-tight configuration while the steam is delivered to the plant fibres, so that steam pressure inside the container increases above atmospheric pressure.

30. A method according to claim 28 or 29, in which the method comprises the step of sensing a pressure inside the container, and/or sensing a humidity inside the container.

31. A method according to claim 28, 29 or 30, comprising the step of controlling the supply of steam to reach a predetermined steam pressure inside the container.

32. A method according to any of claims 28 to 31, comprising the step of opening a valve in the container to vent steam from the interior of the container to the outside atmosphere when the pressure, or the temperature, or the humidity in the container exceeds a predetermined level.

33. A method of defrosting and re-hydrating frozen plant fibres, comprising the method of any of claims 23 to 32,
in which the supply of steam is controlled to raise the temperature of a frozen batch of plant fibres to a target temperature range of between 3° C. and 8° C., preferably between 4° C. and 6° C., or 4° C. and 5° C.

34. A method according to claim 33, in which In which steam is supplied to the batch of plant fibres until the moisture content of the batch of plant fibres is between 8% and 14%, preferably between 10% and 12% moisture content.

35. A method of steam cleaning plant fibres, comprising the method of any of claims 23 to 32,
in which the supply of steam is controlled to raise the temperature of a batch of plant fibres to a target temperature range of between 90° C. and 104° C.

36. A method according to claim 35, in which the supply of steam is controlled to raise the temperature of the batch of plant fibres to a temperature of at least 90° C. for a period of at least 10 minutes, or at least 15 minutes, or at least 20 minutes.

37. A method according to claim 35 or 36, in which the supply of steam is controlled to raise the temperature of the batch of plant fibres to a maximum temperature of 104° C. for a maximum of 30 minutes.

38. An apparatus or method according to any preceding claim, in which the plant fibres are animal fodder, such as hay or haylage, or in which the plant fibres are industrial hemp fibres.
